(19) United States
(12) Patent Application Publication
(10) Pub. No.: US 2018/0209999 A1
(43) Pub. Date: Jul. 26, 2018

(54) AUTOMATIC QUALITY CONTROL DEVICE OF BLOOD CELL ANALYZER

(71) Applicant: THE CATHOLIC UNIVERSITY OF KOREA INDUSTRY-ACADEMIC COOPERATION FOUNDATION, Seoul (KR)

(72) Inventor: Kyung Ja HAN, Gwacheon-si, Gyeonggi-do (KR)

(21) Appl. No.: 15/746,749

(22) PCT Filed: Oct. 28, 2015
(86) PCT No.: PCT/KR2015/011416

§ 371 Date: Jan. 22, 2018

(30) Foreign Application Priority Data
Jul. 31, 2015 (KR) 10-2015-0108454

(51) Int. Cl. G01N 35/00; G01N 35/10; G01N 33/50; G01N 33/487

(52) U.S. Cl. CPC G01N 35/00613; G01N 35/10; G01N 2035/00683; G01N 33/48785; G01N 33/50

(57) ABSTRACT

Provided is a technique for automatically performing quality control of a blood cell analyzer so as to minimize waste of time and manpower and quality control errors through automation of everyday repetitive quality control tasks. According to an embodiment of the present invention, an automatic quality control device of a blood cell analyzer includes: a reagent storage unit installed on the blood cell analyzer and configured to refrigerate and store at least one reagent for use in quality control of the blood cell analyzer; a transfer device installed between the reagent storage unit and a blood cell test unit of the blood cell analyzer for moving the at least one reagent from the reagent storage unit to the blood cell test unit of the blood cell analyzer; and a quality control controller configured to control an operation of the transfer device, receive from the blood cell test unit information about results of a quality control test performed using the at least one reagent to determine whether the quality control test is passed, and output information about results of the determination whether the quality control test is passed through an output device of the blood cell analyzer.

### TECHNICAL FIELD

The present invention relates to a quality control system of a blood cell analyzer, and more particularly, to a technique for minimizing manpower and errors by automatically perform quality control of a blood cell analyzer.

### BACKGROUND ART

Blood cell analyzers are medical devices used in laboratories of all hospitals for complete blood cell counts. Like other test instruments, blood cell analyzers are used for testing only when the blood cell analyzers have passed a quality control test performed before testing specimens of patients, so as to guarantee test accuracy and reliability.

For the quality control of a blood cell analyzer, reagents prepared usually from animal cells and stored in a refrigerator are previously put at room temperature in the morning, and then a quality control test is performed after the preparation of equipment is completed.

If results of the quality control test are normal, it is determined that the preparation of a test is finished, and the blood cell analyzer is used to test real specimens of patients. However, if the results of the quality control test are abnormal, the blood cell analyzer is rinsed, and the quality control test is performed again. If the quality control test is not passed again, an engineer may visit to check the blood cell analyzer.

This quality control is an everyday simple repetitive task consuming a long time as well, and as the number of pieces of equipment increases, much more time and manpower are consumed for quality control, thereby causing problems relating to time and manpower.

To solve these problems, companies such as Beckman Coulter have developed products such as a product configured to control a blood cell analyzer such that the blood cell analyzer may be ready to perform a quality control test at a set time.

The product has a function of automatically rinsing a test unit such that equipment is ready for quality control before a tester comes to work.

### DETAILED DESCRIPTION OF THE INVENTION

### Technical Problem

However, it has been pointed out that such equipment cannot minimize the consumption of time and manpower in quality control because when a quality control test is actually performed, a tester has to place a reagent on a test unit of a blood cell analyzer after the tester takes the reagent from a refrigerator and leaves the reagent at room temperature for 20 minutes to 30 minutes, and the tester has to manually evaluate results of the test performed using the reagent.

Therefore, an objective of the present invention is to provide a technique for automatically performing quality control of a blood cell analyzer so as to minimize waste of time and manpower through automation of everyday repetitive quality control tasks.

In addition, another objective of the present invention is to improve reliability in the performance of a blood cell analyzer by minimizing quality control errors occurring in manual tests.

In addition, another objective of the present invention is to minimize potential problems relating to manpower in quality control tests by automatically perform quality control tests when quality control testers are insufficient.

### Technical Solution

To achieve the above-described objectives, an embodiment of the present invention provides an automatic quality control device of a blood cell analyzer, the automatic quality control device including: a reagent storage unit installed on the blood cell analyzer and configured to refrigerate and store at least one reagent for use in quality control of the blood cell analyzer; a transfer device installed between the reagent storage unit and a blood cell test unit of the blood cell analyzer for moving the at least one reagent from the reagent storage unit to the blood cell test unit of the blood cell analyzer; and a quality control controller configured to control an operation of the transfer device, receive from the blood cell test unit information about results of a quality control test performed using the at least one reagent to determine whether the quality control test is passed, and output information about results of the determination whether the quality control test is passed through an output device of the blood cell analyzer.

### Advantageous Effects of the Invention

As described above, according to the present invention, quality control of the blood cell analyzer may be automatically performed at a set time by using the reagent storage unit, the transfer device, and the quality control controller installed on the blood cell analyzer, thereby minimizing waste of time and manpower through automation of everyday repetitive quality control tasks.

At the same time, when a quality control test is performed, manual test errors may be prevented through automation, and automatic quality control may be possible for the case of insufficient testers, thereby minimizing quality control problems relating to manpower.

### DESCRIPTION OF THE DRAWINGS

FIG. 1 is a schematic view illustrating a configuration of an automatic quality control device of a blood cell analyzer according to an embodiment of the present invention.

FIG. 2 is a flowchart illustrating a functional flow of the automatic quality control device of the blood cell analyzer according to an embodiment of the present invention.

### BEST MODE

Hereinafter, an automatic quality control device of a blood cell analyzer will be described according to embodiments of the present invention with reference to the accompanying drawings.

In the following description, well-known techniques relating to features of the present invention will not be described for a clearer understanding of the present invention. Detained descriptions of the following embodiments are for facilitating understanding of the present invention and are not intended to limit the scope of the present invention. Therefore, equivalent inventions having the same functions as the present invention are included in the scope of the present invention.

In the following description, like reference numbers refer to like elements, and unnecessary repeated descriptions and descriptions of well-known techniques will not be given.

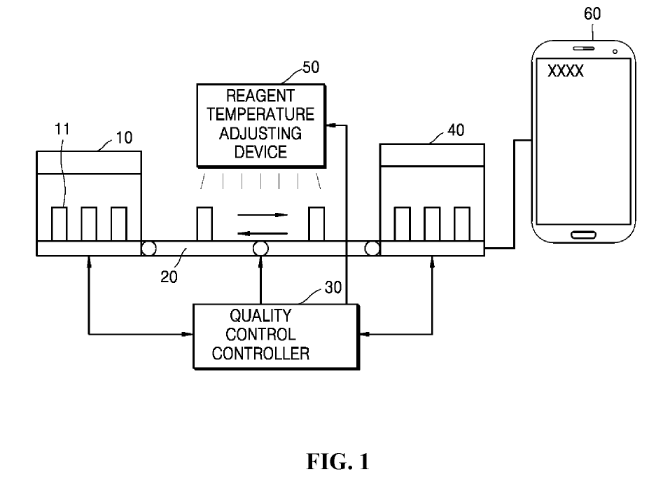

FIG. 1 is a schematic view illustrating a configuration of an automatic quality control device of a blood cell analyzer according to an embodiment of the present invention.

Referring to FIG. 1, according to the embodiment of the present invention, the automatic quality control device of the blood cell analyzer includes a reagent storage unit 10, a transfer device 20, and a quality control controller 30. In addition, the automatic quality control device may further include a reagent temperature adjusting device 50.

The reagent storage unit 10 is a structure additionally installed in a region of the blood cell analyzer so as to refrigerate and store at least one reagent 11 (for example, animal cells) to be used for quality control of the blood cell analyzer.

The reagent storage unit 10 may be manufactured so as to be installed in connection with the blood cell analyzer or be installed in the blood cell analyzer. The at least one reagent 11 to be used for quality control may be provided in the reagent storage unit 10 in the order of use in the quality control.

The transfer device 20 is installed between the reagent storage unit 10 and a blood cell test unit 40 of the blood cell analyzer to transfer the at least one reagent 11 stored in the reagent storage unit 10 to the blood cell test unit 40.

In FIG. 1, the transfer device 20 is illustrated as a simple device such as a transfer track. However, the transfer device 20 may include: a device for opening and closing a door through which the reagent 11 is stored in the reagent storage unit 10 and taken out of the reagent storage unit 10; a device having a forceps shape or another shape for loading or unloading the reagent 11 into or from the reagent storage unit 10 in order; a transfer device such as a track for guiding and controlling transfer of the reagent 11 between the reagent storage unit 10 and the blood cell test unit 40; a device for opening and closing a door through which the reagent 11 is put into and taken out of the blood cell test unit 40; and a device having a forceps shape or another shape for loading or unloading the reagent 11 into or from the blood cell test unit 40 in order.

The reagent temperature adjusting device 50 may be installed at a periphery of the transfer device 20 to perform a temperature adjusting function for changing the reagent 11 from a refrigerated state to a room-temperature state for quality control when the reagent 11 is moved from the reagent storage unit 10 to the blood cell test unit 40.

Preferably, the reagent temperature adjusting device 50 may include: a temperature sensor configured to measure the temperature of the reagent 11 when the reagent 11 is moved by the transfer device 20; and a temperature control device such as a heater and a cooler configured to heat or cool a surrounding area according to measured values of the temperature sensor.

The quality control controller 30 has a function of controlling the operation of the transfer device 20 to transfer the reagent 11 into the blood cell test unit 40 for a quality control test, a function of receiving, from the blood cell test unit 40, information about results of the quality control test performed by the blood cell test unit 40 using the reagent 11 so as to determine whether the quality control test is passed, and a function of outputting information about results of the determination to an output device 60 of the blood cell analyzer.

Specifically, a memory of the quality control controller 30 may store information such as a plurality of time points or periods such that the quality control controller 30 may perform a quality control test at a set time. At a time point stored in the memory, the quality control controller 30 operates the transfer device 20 to transfer at least one reagent 11 to the blood cell test unit 40 for a quality control test.

In addition, after the quality control test, the quality control controller 30 operates the transfer device 20 to transfer the reagent 11 used for the quality control test from the blood cell test unit 40 to the reagent storage unit 10.

In the case of failing to pass the quality control test, as the quality control controller 30 receives, from the blood cell test unit 40, results of the quality control test performed using the reagent 11, the quality control controller 30 determines that the quality control test is not passed. In this case, the quality control controller 30 may operate a rinsing device (not shown) installed on the blood cell test unit 40 so as to rinse the blood cell test unit 40, and may control the transfer device 20 to transfer a reagent 11 again to the blood cell test unit 40 so as to perform a quality control test again.

That is, after the quality control test, the reagent 11 may be transferred to the reagent storage unit 10 by the transfer device 20 under the control of the quality control controller 30. At this time, if the quality control test is not passed, the quality control controller 30 automatically rinses the blood cell test unit 40 and transfers a reagent 11 to the blood cell test unit 40 again so as to perform a quality control test again.

After the quality control test is performed again, if it is determined, based on information on results of the quality control test received from the blood cell test unit 40, that the quality control test is not passed, the quality control controller 30 extracts information about a manager of the blood cell analyzer from the memory, that is, information about a terminal of at least one manager including an engineer who performs a blood cell test using the blood cell analyzer or checks and repairs the blood cell analyzer, and transmits alarming information to the terminal corresponding to the extracted manager terminal information so as to report that it is necessary to check the blood cell analyzer.

In addition, information output through the output device 60 may include information about the lot number, expiration date, and remaining amount of a reagent used in a quality control test as quality control test information in addition to the above-described information about whether the quality control test is passed, such that a user of the blood cell analyzer may check detailed information about quality control.

According to the functions of the automatic quality control device of the blood cell analyzer of the embodiment of the present invention, quality control may be automatically performed at a set time, and actions may be automatically taken according to results of the quality control.

Therefore, waste of time and manpower may be minimized in everyday repetitive quality control tasks, and quality control problems relating to manpower may be solved. In addition, since quality control is automatically performed, errors caused by manual inspection may be minimized when reagents are loaded and tests are performed.

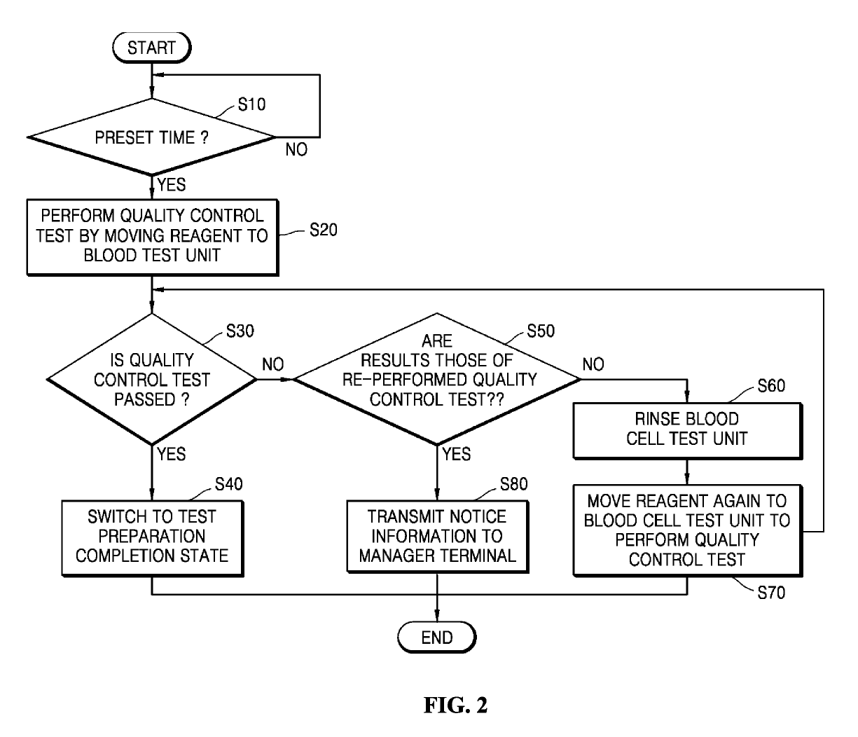

FIG. 2 is a flowchart illustrating a functional flow of the automatic quality control device of the blood cell analyzer according to an embodiment of the present invention. The same descriptions as those given with reference to FIG. 1 will not be repeated in the following description. Unless otherwise described, functions illustrated in FIG. 2 are performed by the quality control controller.

Referring to FIG. 2, the device performs Step S10 to determine whether it is a quality control time, that is, whether it is a test time previously stored in the memory.

If it is determined that it is a previously set time, Step S20 is performed in which a reagent is transferred from the reagent storage unit to the blood cell test unit, and the blood cell test unit performs a quality control test using the reagent.

After Step S20, as described above, the quality control controller performs a function of determining, based on results of the quality control test, whether the quality control test is passed. Thereafter, in Step S30, the quality control controller determines whether the quality control test is passed. If it is determined that the quality control test is passed, the blood cell analyzer is switched to a test preparation completion state in Step S40, and thus the blood cell analyzer is ready to use.

Meanwhile, in Step S30, if it is determined that the quality control test is not passed, the device performs Step S50 to determine whether the quality control test not passed is performed because a previous quality control test was not passed. That is, it is determined whether the quality control test not passed is a test performed for the first time or for the second time after the first quality control test was not passed and thus the blood cell test unit was rinsed.

In Step S50, if it is determined that the quality control test is performed for the first time and is not passed, the device performs Step S60 to rinse the blood cell test unit and then Step S70 to transfer a reagent to the blood cell test unit and perform a quality control test again.

However, in Step S50, it is determined that the quality control test not passed is a test performed after failure to pass and rinsing of the blood cell test unit, as described above, the device performs Step S80 to transmit alarming information (notice information) to a terminal of a manager such that the blood cell analyzer may be checked.

In the above descriptions of the embodiments, although all the elements are described as being combined as one entity or operating in combination, the present invention is not limited to the embodiments. For example, all the elements may be selectively combined as at least one entity and may then be operated within the scope of the present invention.

Furthermore, in the above description, the terms “comprises,” “constituted by” and/or “have” specify the presence of stated elements, but do not preclude the presence or addition of one or more other elements unless otherwise specified. Unless defined otherwise, all terms used herein, including technical and scientific terms, have the same meanings as those commonly understood by those skilled in the art to which the present invention pertains. Terms such as those defined in a generally used dictionary may be interpreted to have the same meanings as the contextual meanings in the relevant field of art, and are not to be interpreted to have ideal or excessively formal meanings unless clearly defined herein.

The above description of the present invention is for illustrative purposes only, and it will be apparent to those skilled in the art that modifications and variations could be made without departing from the scope of the present invention. That is, the embodiments of the present invention are for illustrative purposes only and are not intended to limit the scope of the present invention. Therefore, the scope of the present invention should be construed according to the appended claims, and it should be understood that all technical ideas equivalent to those described above are within the scope of the present invention.

1. An automatic quality control device of a blood cell analyzer, the automatic quality control device comprising:
a reagent storage unit installed on the blood cell analyzer and configured to refrigerate and store at least one reagent for use in quality control of the blood cell analyzer;
a transfer device installed between the reagent storage unit and a blood cell test unit of the blood cell analyzer for transferring the at least one reagent from the reagent storage unit to the blood cell test unit of the blood cell analyzer; and
a quality control controller configured to control an operation of the transfer device, receive from the blood cell test unit information about results of a quality control test performed using the at least one reagent to determine whether the quality control test is passed, and output information about results of the determination whether the quality control test is passed through an output device of the blood cell analyzer.

2. The automatic quality control device of claim 1, wherein the quality control controller is configured to operate the transfer device at a time previously stored in a memory of the quality control controller so as to transfer the at least one reagent to the blood cell test unit.

3. The automatic quality control device of claim 1, wherein after the quality control test, the quality control controller operates the transfer device to transfer the at least one reagent used for the quality control test from the blood cell test unit to the reagent storage unit.

4. The automatic quality control device of claim 3, wherein if the quality control controller determines that the quality control test is not passed based on the information, received from the blood cell test unit, about the results of the quality control test performed using the at least one reagent, the quality control controller operates a rinsing device installed on the blood cell test unit to rinse the blood cell test unit and operates the transfer device to transfer a reagent again to the blood cell test unit such that a quality control test is re-performed by the blood cell test unit.

5. The automatic quality control device of claim 4, wherein after the blood cell test unit re-performs the quality control test, if the quality control controller receives information about results of the quality control test and determines that the quality control test is not passed, the quality control controller extracts previously-stored information about a terminal of a manager of the blood cell analyzer and transmits alarming information to the terminal corresponding to the extracted information to report that the blood cell analyzer has to be checked.

6. The automatic quality control device of claim 1, further comprising a reagent temperature adjusting device installed at a periphery of the transfer device so as to change the at least one reagent to a room-temperature state while the at least one reagent is transferred to the blood cell test unit.

7. The automatic quality control device of claim 1, wherein the quality control controller controls the output device of the blood cell analyzer such that at least one of a lot number, an expiration date, and a remaining amount of the at least one reagent is output as quality control test information through the output device.
